(19) United States
(12) Patent Application Publication
(10) Pub. No.: US 2025/0387067 A1
(43) Pub. Date: Dec. 25, 2025

(54) System and Method for Enhancing Visual Accuracy and Catheter Manipulation During Cardiac Electrophysiology Mapping and Ablation Procedures Using 3D Visualization Technology

(71) Applicant: Basil Abu-El-Haija, San Jose, CA (US)

(72) Inventor: Basil Abu-El-Haija, San Jose, CA (US)

(21) Appl. No.: 19/282,896

(22) Filed: Jul. 28, 2025

(51) Int. Cl. A61B 5/367; A61B 5/00; A61B 18/00; A61B 18/14; A61B 34/10; A61B 90/00

(52) U.S. Cl. CPC A61B 5/367; A61B 5/742; A61B 18/1492; A61B 34/10; A61B 2018/00351; A61B 2018/00577; A61B 2034/105; A61B 2090/365; A61B 2090/367; A61B 2090/368

(57) ABSTRACT

A system and method for enhancing visual accuracy comprising a computer processor and memory that executes software to capture the 3D map of the heart images, heart valves, and electro-anatomic data of the heart chambers, and project it into a 3D monitor instead of a 2D monitor, where in the displayed 3D map of the heart images, heart valves, heart chambers anatomy and catheters, is still rendered as a 2D map, which then can be visualized as a real 3D images and map by using/wearing special glasses (3D glasses) designed to view the 3D map of the heart chambers and catheters as 3D in real time.

### BACKGROUND OF THE INVENTION

Ablation procedures are the treatment of many cardiac rhythm disorders. The procedure starts by obtaining a three-dimensional (3D) map of the heart chambers and catheters used to obtain the 3D map and for ablation, which is a 3D representation of each patient's heart chamber(s); by introducing catheters into the heart, through a big vein, like the femoral vein in the lower body, to the heart chambers, which then collect the information needed to build a 3D electro-anatomic map-3D map-of any heart chamber (3D mapping of the heart chambers). When the introduced mapping catheters touch any area inside the heart chamber, that area gets collected to build the 3D electro-anatomic map of the heart chambers. The 3D map of the heart chamber of interest that the operator collects during mapping gets acquired while it is being viewed on a 2D monitor. The 3D map of the heart chamber during the acquisition of the map gets displayed on a 2D display and viewed as 2D map. An operator visualizes the mapping catheters inside the heart chamber on a 2D display to help guide an operator for the exact location and for the maneuvering of the catheters inside the heart chamber(s). After acquiring the 3D map of the heart chambers, ablation at the target areas in the heart chambers begins by an operator to treat the areas that need ablation by introducing ablation catheters to the heart (through a femoral vein or other big veins) that project into the 3D map of the heart chamber of interest that the operator collects during mapping while viewing the 3D map of the heart chamber during the ablation of the 3D cardiac structure or tissue on a 2D display. Many challenges are encountered during the procedures because the heart is a complex 3D structure, and each chamber of the heart has its own anatomic characteristics and challenges occur when maneuvering the catheters inside the heart chambers while being viewed on a 2D display.

Current Cardiac electrophysiology procedures, utilizing cardiac 3D mapping systems for creating a 3D map of the heart chambers of interest, and ablation of arrhythmias, rely heavily on two-dimensional (2D) visual interfaces to interpret complex three-dimensional (3D) anatomical data, even though the operator utilizes catheters designed to obtain a 3D map of any heart chamber to collect anatomy, electrical activity, activation time and voltage (electro-anatomic map or 3D map). To collect anatomy, electrical activity, activation time and voltage associated with each anatomic locations, including each and any point of the heart, is done by moving and manipulation of the mapping catheters inside the heart chambers while viewing the 3D map that an operator collects using the mapping catheter on a 2D display, which can limit the operator's understanding of the exact and precise location of the catheters as well as each area in the heart or points that were collected by the mapping system catheters during cardiac chambers 3D mapping and ablation procedures. The current cardiac chambers 3D mapping systems also depend on mapping specialists who participate in the procedure of 3D mapping and ablation of cardiac chambers, who can tilt and rotate the map of the heart chambers, that an operator is collecting by the mapping catheter(s) inside the heart chamber of interest, in multiple angles, as needed, during the acquisition of the 3D map of the heart chamber to help the operator understand their catheters' anatomic locations. This tilting of the image occurs at the request of the mapping physician during acquisition of the 3D map of the heart chambers and during ablation that occurs after obtaining the map.

These limitations (displaying a 3D anatomic structure into a 2D monitor) can reduce spatial accuracy, more difficulty in catheters manipulation inside the heart, prolong procedure times, and increase operator fatigue, in addition to more redo operations, reduce the precise placement location of ablation catheters inside the heart chambers, and more procedure related complications that can result from inappropriate movement of the catheters inside the heart chambers and the force applied to the catheter while moving it inside the heart chambers, which can cause damage to heart chamber structures or placement of ablation therapy in a location that can cause harm to the patients. Current mapping and ablation systems do not adequately provide immersive, real-time 3D visualization to a 3D monitor that mirrors the anatomical complexity of the heart, that can be viewed utilizing special glasses designed for 3D monitors.

There is a need for an improved visual interface that enhances operator perception of the cardiac chamber anatomy and catheters' precise locations inside the heart chambers during cardiac electrophysiology 3D mapping of cardiac chambers and ablation procedures, particularly depth and spatial orientation, to facilitate catheter manipulation and ablation at the target lesion resulting in more accurate and efficient procedures with less complications.

### DESCRIPTION OF THE INVENTION

The present invention provides a novel system and method that incorporates three-dimensional (3D) monitors and compatible 3D glasses into the procedural workflow of cardiac electrophysiology 3D mapping of cardiac chambers and ablation procedures (creating a 3D map of the heart chambers of interest and ablation of the area inside the heart chamber). The invention can be also applied to any cardiac imaging modality that builds 3D models of the heart and all other heart structures (valves, arteries, etc) into 2D displays as in cardiac imaging with ultrasound that builds 3D models of all heart structures, like heart chambers, valves, arteries, and veins. Cardiac Magnetic Resonance Imaging (MRI) and Cardiac Computed Tomography (CT) also display the 3D image of the heart and any existing image of the hearts' structures into a 2D display.

The system and method enhance spatial resolution and depth perception of electro-anatomical maps (3D maps of the heart chambers) and ablation targets in the heart chambers. Providing compatibility layers for integration with any existing mapping systems that are used to obtain a 3D map of the heart chambers and ablation, and utilizes real-time rendering of cardiac structures in a 3D format.

Operators view these enhanced visuals using passive or active 3D glasses, allowing more intuitive catheter navigation inside the heart chambers and ablation lesions placement. The system and method include features such as adjustable viewing angles, head tracking, or Augmented Reality (AR) overlays to further improve spatial awareness.

This invention integrates with any existing cardiac mapping technologies and systems to create a 3D map of the heart chamber that is being displayed and projected into a 2D monitor, for the diagnosis and treatment of cardiac arrhythmias by creating a 3D map of the heart chamber of interest, with a 3D visualization display (instead of a 2D display) that can be viewed through 3D glasses. This innovative approach allows the operator to perceive complex cardiac structures and electrical pathways in a more intuitive manner, facilitating precise maneuvering of the catheters and ablation of arrhythmias.

Since the described 3D visualization system can enhance the operator's ability to navigate the cardiac anatomy or the cardiac chamber that is needing mapping (i.e., creating a 3D representation of the heart chambers) and ablation, it will reduce the complications of ablation procedures significantly and improve the success rate of ablating at the correct site/area of heart tissue that needs ablation, and target specific arrhythmic areas inside the heart chambers more effectively. This is in addition to avoid damaging important anatomic structures inside the heart chambers. This will also result in unnecessary extra ablations (that the operator might do because of the limitations of viewing the exact location in a 2D display) that can further increase the complications, and decrease the success rate of ablation if ablation is done near but not at the exact location (by creating edema in the area which subsequently results in less durable lesions). Live 3D visualization while manipulating and moving any catheter inside the heart would make such movements much easier and add more accuracy, which will help aid the operator understanding of the geometry and anatomy of the heart chamber that is being collected (3D map) then targeted for ablation.

### DETAILED DESCRIPTION OF THE INVENTION

The invention of this visualization system consists of a three-dimensional (3D) monitor capable of stereoscopic 3D display using polarization or shutter technology pair of 3D glasses (passive or active) worn by an operator. This visualizations system for enhancing visual accuracy comprising of a computer processor and memory that execute and process software to capture the 3D map of the heart chambers and catheters, and electro-anatomic data of the heart chambers, and project it into a 3D monitor instead of a 2D monitor, where in the displayed 3D map of the heart chambers and catheters, is still rendered as a 2D map, which then can be visualized as a real 3D map of the heart chambers and catheters by using/wearing special glasses (3D glasses) designed to view the 3D map of the heart chambers and catheters as 3D in real time on a 3D display.

Incorporating this visual system (3D map) into any existing and current systems used for 3D cardiac chambers mapping and ablation procedures for cardiac chambers in cardiac electrophysiology procedures for the treatment of heart rhythm disorders, so that the 3D map (of the heart chamber of interest) that an operator collects using any existing cardiac chambers 3D mapping systems, gets displayed into a 3D monitor instead of a 2D monitor. This 3D map of the heart chamber can then be visualized by the operator using special glasses to visualize structures as real 3D structures and in real time. This visualization system will result in enhancing visual accuracy of the 3D map of the heart chambers as well as the catheters used for obtaining the 3D map of the heart chambers and the catheters used for ablation of the heart chamber areas (catheters) during catheter manipulation in the heart chambers during cardiac electrophysiology procedures for mapping and ablation of heart chambers to treat certain heart rhythm disorders.

The invention allows the currently available and any existing cardiac chambers mapping and ablation systems, that are used to create a 3D map of the heart chambers during cardiac electrophysiology procedures to treat heart rhythm disorders with ablation, generate a 3D map that gets projected on a 2D display, to use this 3D visualization technology. This map gets built by the operator by using mapping catheters that go inside the heart and the map is built when the catheter touches the heart chamber (the left atrium of the heart as an example) from the inside. FIG. 1 shows an example of a 3D map of the left atrium of the heart obtained by current cardiac mapping systems by moving the mapping catheters inside the left atrium of the heart that gets projected into a 2D display showing anatomy, electrical activity, activation time and voltage associated with each anatomic locations, including each and any point that the mapping catheter touched.

Each area that gets touched by the catheters gets collected and projected as a 3D model that is displayed on 2D monitors (FIG. 1). The invention is to use any existing cardiac chambers mapping systems that build 3D maps of the heart chambers to project the acquired 3D map into a 3D monitor instead of a 2D monitor, in addition to having operators wear special glasses (3D glasses) so operators can see the 3D map and the catheters that are being moved and manipulated inside the heart in real time in 3D, so that the exact location of the catheters and areas of interest in the heart chambers, where ablation should occur, get viewed by the operator in a 3D view instead of a 2D view. FIG. 2 is an example of a 3D map of the left atrium of the heart showing the catheter that is being manipulated by an operator, such catheter can be used for mapping and ablation.

Electroanatomic maps (3D maps of the heart chambers) to help operators visualize a 3D map of the heart as generated by an electro-anatomical mapping systems used in cardiac electrophysiology (EP) studies. These systems create color-coded 3D reconstructions of heart chambers (usually atria or ventricles), showing things like:
1. Voltage maps (to detect scar tissue)
2. Activation maps (to track electrical impulse timing)
3. Propagation maps (to visualize electrical wavefront movement)

FIG. 3 is an example of a 3D map that is tilted by a mapping specialist to help operators visualize catheters from the inside to help and guide further maneuvering of the catheters inside the heart chamber

A head tracking module to adjust perspective dynamically based on operator head position can also be added to the system. This head tracking module can then adjust the view angle based on the operator head movements (can sense operators head movement).

The invention consists of a adding a 3D monitor that displays the built 3D electroanatomic map (3D map of the heart chambers and catheters) in real time and wearing special 3D glasses to view 3D monitors display in 3D (voltage at that area of the heart at an activation time that is calculated) to be added and incorporated to any existing cardiac chambers 3D mapping and ablation systems and the currently United States Food and Drug Administration (FDA) approved anatomic cardiac chambers mapping and ablation systems for cardiac chamber 3D mapping and ablation of arrhythmias in the heart chambers, comprising a mapping catheter with electrode arrays for detecting electrical signals, in real time, a data processing unit and memory for generating a 3D map of cardiac anatomy and electrical activity (including the current and any existing cardiac chambers 3D mapping and ablation systems. This system and method for enhancing visual accuracy includes a computer processor and memory that executes software to capture the 3D map of the heart chambers and catheters, and electro-anatomic data of the heart chambers, and project it into a 3D monitor instead of a 2D monitor, and 3D glasses so that the operator can view the 3D map in real time 3D.

Adding three dimensional (3D) monitors instead of two-dimensional (2D) monitors that can be incorporated with any existing cardiac chambers mapping and ablation systems used in cardiac electrophysiology procedures for 3D mapping of the heart chambers (3D mapping) using mapping catheters; and ablation of certain areas in the heart chambers using ablation catheters. The creation of the 3D map of the heart chamber(s) of interest (3D mapping of heart chambers) is done by operators who can then visualize the catheters used for and during 3D mapping of the heart chambers and ablation (catheters) in addition to the 3D map of the heart chambers that gets collected, by using special 3D glasses to visualize the 3D map of the heart chambers and the catheters as real 3D structures, during acquisition of the 3D map of the heart chambers and during maneuvering the catheters used for mapping of the heart chambers and the catheters used during ablation (ablation catheters) of a specific area of the heart chamber(s) to treat certain heart rhythm disorders.

The benefits of having a 3D visualization system that gets incorporated into the current cardiac mapping systems are enormous. The benefits include improved spatial awareness of the heart's structure, enhanced precision in targeting arrhythmic sites in the heart chambers using the ablation catheters while viewing in real time 3D, obtaining a more detailed 3D map of the heart chambers by better understanding of the exact location of the mapping catheters, easier manipulation of mapping and ablation catheters which reduces complications related to improper catheter manipulation and force applied to the catheters that can damage normal heart chambers structures and valves, and reduced procedure time and fluoroscopy time which result in less radiation exposure to the patient as well as the operators and other supporting staff (nurses, anesthesiologists, x-ray operators, etc.). Having more precision and awareness of the heart chamber geometry by viewing it in real time 3D, will result in more precise manipulation of the catheters inside the heart chambers to target ablation locations precisely, which will lead to improved patient outcomes and procedure success rates, decrease complications related to the procedure and to the ablation part of the procedure specifically, in addition to decreased re-operation rate (decrease in repeat procedures for ablation of the same arrhythmia in the same patient).

This visual enhancement system can be also applied to other areas of the imaging of the heart chambers, and not only to Cardiac Electrophysiology procedures described above. For example, this visual enhancement system can be applicable to cardiac ultrasound (3D images obtained) using either transthoracic cardiac ultrasound or transesophageal cardiac ultrasound of the heart chambers, cardiac Magnetic Resonance Imaging (MRI), cardiac Computed tomography (CT) scans.

Patient's privacy will be protected and will be treated according to the accepted workplace rules and standards and follow the federal and state laws and regulations related to keeping patients' privacy protected all the time.

### BRIEF DESCRIPTION OF THE DRAWINGS

FIG. 1: Example of a 3D map of the left atrium of the heart obtained by moving the mapping catheters inside the left atrium of the heart projected into a 2D display showing anatomy, electrical activity, activation time and voltage associated with each anatomic locations, including each and any point that the mapping catheter touched.

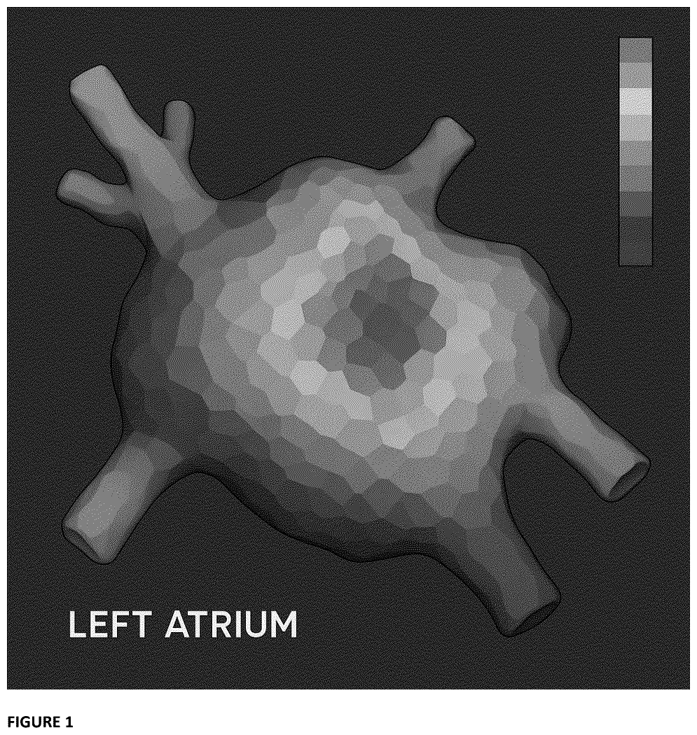

FIG. 2: Discloses 3D map of the left atrium showing the catheter that an operator moves for mapping and ablation.

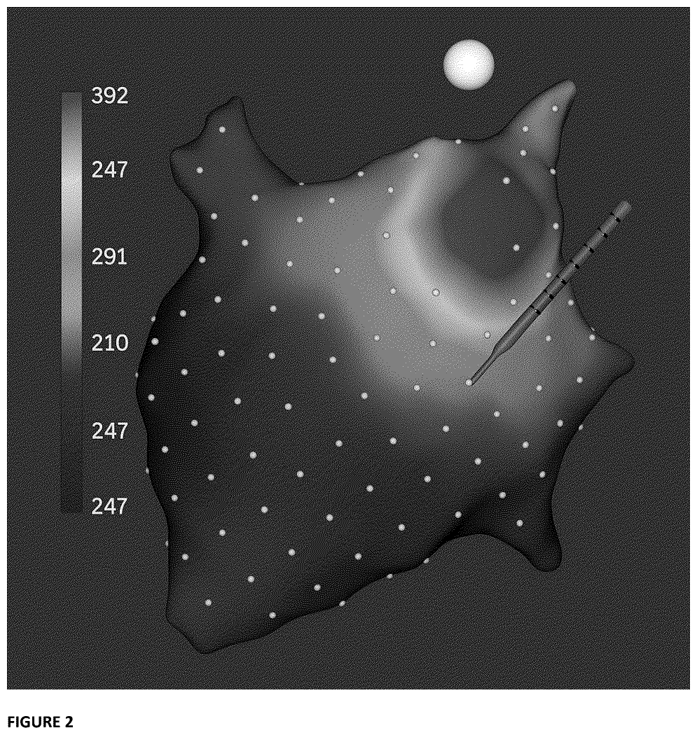

FIG. 3: A 3D map of the left atrium of the heart tilted to help an operator visualize the catheter from the inside to help an operator visualize the location of the catheter, which will guide further maneuvering of the catheter.

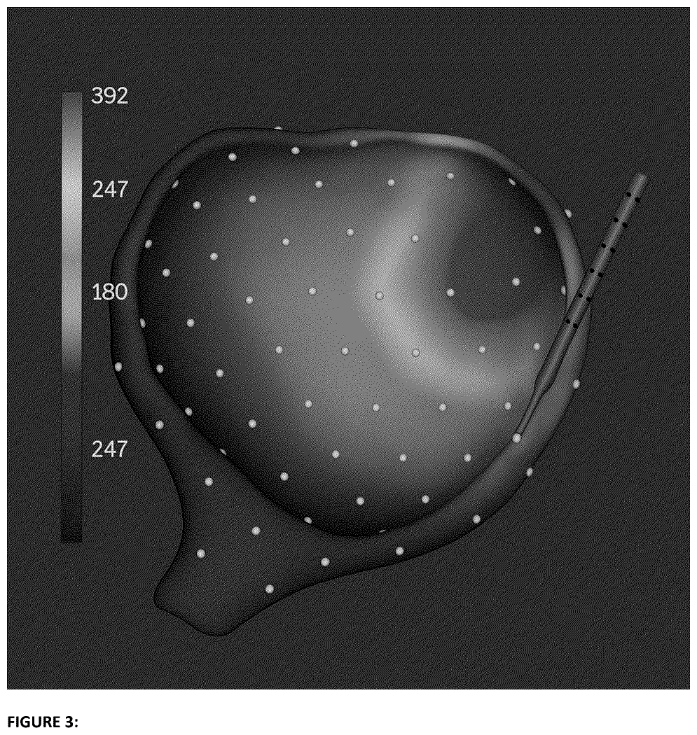

1. A system for enhancing visual accuracy of the three dimensional (3D) map of the heart chambers and catheters comprising: a 3D monitor; a pair of 3D glasses; a processor unit; a memory unit; converting electro-anatomical mapping inputs from any current or any existing mapping systems from a 2D map to a 3D map of the heart chambers and catheters used for 3D mapping and ablation of the heart chambers and catheters during cardiac electrophysiology procedures; projecting the 3D map into the 3D monitor where in the projection is viewed as a two-dimensional (2D) map; wearing 3D glasses to render the 2D map as 3D map to enhance visual accuracy of the heart chambers and their anatomy and the catheters used during mapping and ablation procedures and improve spatial awareness of the catheters during catheters movements and manipulation and the heart chambers anatomy during cardiac electrophysiology 3D cardiac chamber mapping and ablation procedures.

2. The system of claim 1, wherein the 3D monitor is compatible with passive polarized or active shutter-based 3D glasses.

3. The system of claim 1, further comprising a compatibility layer for integration of the 3D map with any existing electro-anatomical mapping systems for creating a 3D map of the heart chambers (3D mapping) using catheters for mapping of the heart chambers (mapping catheters), during cardiac electrophysiology procedures, when mapping of specific areas in the heart chambers occurs, then ablation using ablation catheters to treat arrhythmias and arrhythmic locations in the heart chambers.

4. The system of claim 1, further comprising a head tracking system used to adjust the 3D map of the heart chambers based on an operator's viewpoint, wherein the said head tracking system is used to adjust the 3D map of the heart chambers and catheters based on the angle that the operator changes while moving their head to visualize the 3D map of the heart chambers and the catheters being used while acquiring the 3D map of the heart chamber and during ablation of the heart chamber tissues in cardiac electrophysiology 3D cardiac chambers mapping and ablation procedures that are the treatment of arrhythmias and heart rhythm disorders.

5. The system of claim 1, wherein said spatial awareness of the catheters and heart chambers is improved by the use of Augmented Reality (AR) overlays to further improve spatial awareness of the heart chambers and catheters (mapping and ablation catheters) during cardiac electrophysiology 3D cardiac chambers mapping and ablation procedures that are the treatment of arrhythmias and heart rhythm disorders.

6. The system of claim 1, enhances visual accuracy of the 3D electro-anatomical maps of the heart chambers during cardiac electrophysiology cardiac chamber 3D mapping and ablation procedures using the current available and any existing anatomical 3D cardiac chamber mapping and ablation systems that create a color-coded 3D reconstruction (3D cardiac chamber mapping) of the heart chambers including Voltage maps, Activation maps, and propagation maps, in addition to enhancing the visual accuracy of the catheters used for 3D mapping and ablation.

7. The system of claim 1, wherein viewing the 3D map in real 3D reduces the time of cardiac electrophysiology 3D mapping of heart chambers and ablation procedures, improves ablation therapy placement accuracy in the exact area of the heart chamber using ablation catheters, improves the heart chambers anatomy visualization, reduce procedure related complications, reduce radiation exposure, facilitates catheters manipulation, reduces operator error and the complications related to wrong catheters maneuvering that can cause damage to structures inside the heart chambers, reduces incorrect placement of ablation therapy in the heart chamber of interest, which will all lead to higher success rates of the ablation procedures for cardiac rhythm disorders with less undesirable complications.

8. A computer implemented method for enhancing visual accuracy of the three dimensional (3D) map of the heart chambers and catheters comprising: convert electro-anatomical mapping inputs from any current or any existing mapping systems from a 2D map to a 3D map of the heart chambers and catheters used for mapping of the heart chambers and catheters used for ablation during ablation procedures of heart chambers' specific areas; project the 3D map into the 3D monitor where in the projection is viewed as a two-dimensional (2D) map; wear 3D glasses to render the 2D map as 3D map to enhance visual accuracy of the heart chambers and their anatomy, and the catheters used during mapping and ablation procedures to enhance spatial awareness of the catheters during catheters movements and manipulation, and the heart chambers anatomy during cardiac electrophysiology mapping and ablation procedures.

9. The computer implemented method in claim 8, wherein the 3D monitor is compatible with passive polarized or active shutter-based 3D glasses.

10. The computer implemented method in claim 8, further comprising a compatibility layer for integration of the 3D map with any existing electro-anatomical mapping systems for creating a 3D map of the heart chambers (3D mapping) using catheters for mapping of the heart chambers (mapping catheters), during cardiac electrophysiology procedures, where mapping and ablation of specific areas in the heart chambers occurs, using ablation catheters, to treat arrhythmias and arrhythmic locations in the heart chambers.

11. The computer implemented method in claim 8, further comprising a head tracking system used to adjust the 3D map of the heart chambers based on an operator's viewpoint, where in the said head tracking system used to adjust the 3D map of the heart chambers and catheters based on the angle that the operator changes while moving their head to visualize the 3D map of the heart chambers and the catheters being used during acquiring the 3D map of the heart chambers and during ablation of the heart chamber tissues.

12. The computer implemented method in claim 8, wherein said spatial awareness of the catheters and heart chambers is improved by the use of Augmented Reality (AR) overlays to further improve spatial awareness of the heart chambers and catheters (mapping and ablation catheters).

13. The computer implemented method of claim 8, enhances visual accuracy of the 3D electro-anatomical maps of the heart chambers during cardiac electrophysiology cardiac chamber 3D mapping and ablation procedures using the current available and any existing anatomical 3D cardiac chamber mapping and ablation systems that create a color-coded 3D reconstruction (3D cardiac chamber mapping) of the heart chambers including Voltage maps, Activation maps, and propagation maps, in addition to enhancing the visual accuracy of the catheters used for 3D mapping and ablation.

14. The computer implemented method in claim 8, wherein viewing the 3D map in real 3D reduces procedure time of cardiac electrophysiology 3D mapping of heart chambers and ablation procedures, improves ablation therapy placement accuracy in the exact area of the heart chamber using ablation catheters, improves the heart chambers anatomy visualization and understanding of the exact location of the ablation targets of the heart chambers tissue, less procedure related complications, less radiation exposure, easier catheters manipulation, reduces operator error and the complications related to wrong catheters maneuvering that can cause damage to structures inside the heart chambers, reduces incorrect placement of ablation therapy location, which will all lead to higher success rates of the ablation procedures for cardiac rhythm disorders with less undesirable complications.
